(19) United States
(12) Patent Application Publication
(10) Pub. No.: US 2005/0250803 A1
(43) Pub. Date: Nov. 10, 2005

(54) Combination of dopamine agonists and monoamine reuptake inhibitors

(75) Inventors: Paul Glue, Flemington, NJ (US); Mario D. Saltarelli, Lake Bluff, IL (US); Gerard Marek, Indianapolis, IN (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/991,037

(22) Filed: Nov. 17, 2004

Related U.S. Application Data
(60) Provisional application No. 60/525,470, filed on Nov. 26, 2003.

(51) Int. Cl. A61K 31/4745

(52) U.S. Cl. 514/291; 514/292

(57) ABSTRACT

This invention is directed to pharmaceutical compositions and kits comprising (i) a dopamine agonist of the formula II as described in the specification, (ii) a monoamine reuptake inhibitor or pharmaceutically acceptable salt thereof; and optionally (iii) a pharmaceutically acceptable carrier. This invention further relates to methods of treatment using those pharmaceutical compositions. Disorders or conditions that may be treated by the compositions, kits and methods of the invention include hypertension, depression, generalized anxiety disorder, phobias, posttraumatic stress syndrome, avoidant personality disorder, premature ejaculation, eating disorders, obesity, chemical dependencies, cluster headache, pain, Alzheimer's disease, obsessive-compulsive disorder, panic disorder, memory disorders, Parkinson's diseases, endocrine disorders, vasospasm, cerebellar ataxia, gastrointestinal tract disorders, negative symptoms of schizophrenia, premenstrual syndrome, Fibromyalgia Syndrome, stress incontinence, Tourette syndrome, trichotillomania, kleptomania, male impotence, cancer, chronic paroxysmal hemicrania, headache and a combination thereof in a mammal such as a human.

The entire disclosure of parent application U.S. Ser. No. 60/525,470 filed Nov. 26, 2003 is fully incorporated herein by reference thereto.

### BACKGROUND OF THE INVENTION

This invention is directed to pharmaceutical compositions comprising (i) a dopamine agonist of formula II as described herein or a pharmaceutically acceptable salt thereof and (ii) a monoamine reuptake inhibitor, such as a serotonin, norepinephrine, or dopamine reuptake inhibitor, or a combination thereof, or a pharmaceutically acceptable salt of the monoamine reuptake inhibitor; to a kit comprising the same; and to methods of treatment with such compositions and kits.

Selective and mixed monoamine reuptake inhibitors have been used to treat disorders or conditions such as depression, as described, for example, in WO 94/00047 and in Expert Opin. Invest. Drugs, 12(1) (2003), 65-86.

Dopamine agonists exhibit positive activity against disorders or conditions such as Parkinson's disease; depression, including major depression, as described, for example, in Depress. Anx., Vol. 11, pp. 58-65 (2000), and Pharmacopsychiatry, Vol. 34, pp. 137-141 (2001).

### SUMMARY OF THE INVENTION

This invention is directed to a pharmaceutical composition useful for example for treating a disorder or condition selected from the group consisting of hypertension, depression, generalized anxiety disorder, phobias, posttraumatic stress syndrome, avoidant personality disorder, sexual dysfunction, eating disorders, obesity, chemical dependencies, cluster headache, pain, Alzheimer's disease, obsessive-compulsive disorder, panic disorder, memory disorders, Parkinson's diseases, endocrine disorders, vasospasm, cerebellar ataxia, gastrointestinal tract disorders, negative symptoms of schizophrenia, premenstrual syndrome, Fibromyalgia Syndrome, stress incontinence, Tourette's syndrome, trichotillomania, kleptomania, male impotence, cancer, chronic paroxysmal hemicrania, headache and a combination thereof in a mammal, which pharmaceutical composition comprising: (i) a dopamine agonist of the formula II 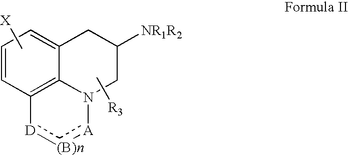
or a pharmaceutically acceptable salt thereof,
wherein R₁, R₂, and R₃ are each independently hydrogen, C₁₋₆ alkyl, C₂₋₆ alkenyl, or C₂₋₆ alkynyl, C₃₋₁₀ cycloalkyl, or R₁ and R₂ are joined to form together with the nitrogen of NR₁R₂ a C₃₋₇ cyclic amine which can contain in addition to said nitrogen one or more heteroatoms selected from the group consisting of N, S and O; X is hydrogen, C₁₋₈ alkyl, halogen, hydroxy, C₁₋₆ alkoxy, cyano, carboxamide, carboxyl, or C₁-C₆ alkoxycarbonyl; A is SO₂, N, CH, CH₂, CHCH₃, C═O, C═S, CSCH₃, C═NH, CNH₂, CNHCH₃, CNHCOOCH₃, or CNHCN; and B is CH₂, CH, C═O, N, NH or N—CH₃; n is 0 or 1; and D is CH, CH₂, C═O, O, N, NH or N—CH₃; (ii) a monoamine reuptake inhibitor or pharmaceutically acceptable salt thereof; and optionally (iii) a pharmaceutically acceptable carrier.

The present invention also relates to:
a method for treating a disorder or condition as described in the previous paragraph in a mammal, comprising administering to a mammal in need of such treatment components (i) and (ii) as described herein;
a pharmaceutical composition for treating a disorder or condition that can be treated by enhancing monoaminergic neurotransmission in a mammal, comprising components (i), (ii), and optionally (iii) as described herein;
a method for treating a disorder or condition that can be treated by enhancing monoaminergic neurotransmission in a mammal, comprising administering to a mammal in need of such treatment components (i) and (ii) as described herein;
a method for enhancing monoaminergic neurotransmission in a mammal, comprising administering to a mammal in need of such treatment components (i) and (ii) as described herein;
a pharmaceutical composition for treating a disorder or condition as described in the previous paragraph in a mammal, consisting essentially of components (i) and (ii) and optionally (iii) as described herein;
a pharmaceutical composition for treating a disorder or condition that can be treated by enhancing monoaminergic neurotransmission in a mammal, consisting essentially of components (i), (ii), and optionally (iii) as described herein;
a kit comprising: a) at least one dopamine agonist unit dosage form of a dopamine agonist of formula II or a pharmaceutically acceptable salt thereof and a pharmaceutically acceptable carrier; b) at least one monoamine reuptake inhibitor unit dosage form comprising a monoamine reuptake inhibitor or a pharmaceutically acceptable salt of said reuptake inhibitor and a pharmaceutically acceptable carrier; and optionally c) a container; and
a kit comprising: a) at least one dopamine agonist unit dosage form comprising a unit dose of a dopamine agonist of formula II or a pharmaceutically acceptable salt thereof, a unit dose of a monoamine reuptake inhibitor or a pharmaceutically acceptable salt of said reuptake inhibitor, and a pharmaceutically acceptable carrier; and optionally b) a container.

### DETAILED DESCRIPTION OF THE INVENTION

“Enhancing monoaminergic neurotransmission,” as used herein, refers to increasing or improving the neuronal process whereby the monoamine serotonin, norepinephrine, dopamine, or a combination thereof is released by a pre-synaptic cell upon excitation and crosses the synapse to stimulate or inhibit the post-synaptic cell.

“Chemical dependency,” as used herein, means an abnormal craving or desire for, or an addiction to a drug. Such drugs are generally administered to the affected individual by any of a variety of means of administration, including oral, parenteral, nasal or by inhalation. “Treating a chemical dependency,” as used herein, means reducing or alleviating such dependency.

A “unit dosage form” as used herein is any form that contains a unit dose of the dopamine agonist of formula II or a pharmaceutically acceptable salt thereof, of the monoamine reuptake inhibitor or a pharmaceutically acceptable salt thereof, or of the dopamine agonist of formula II or pharmaceutically acceptable salt thereof and the monoamine reuptake inhibitor or pharmaceutically acceptable salt thereof. A unit dosage form may be, for example, a tablet or a capsule. A unit dose may be an amount which may be predetermined, for example, by a physician.

A “monoamine reuptake inhibitor” as used herein is a reuptake inhibitor of the monoamine serotonin, norepinephrine, dopamine or a combination thereof. Accordingly, exemplary embodiments of monoamine reuptake inhibitors include serotonin reuptake inhibitors, norepinephrine reuptake inhibitors, and dopamine reuptake inhibitors. The monoamine reuptake inhibitor may have additional pharmacological properties, for example, antagonism of 5-HT_{1A} or 5-HT_{2A/C} receptors. Monoamine reuptake inhibition is readily determined by those skilled in the art according to standard assays.

Exemplary monoamine reuptake inhibitors which may be used in accordance with this invention include those having structure I shown below. 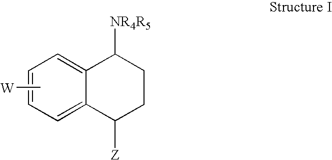
or a pharmaceutically acceptable salt thereof,
wherein R₄ is selected from the group consisting of hydrogen and C₁-C₃ alkyl, R₅ is C₁-C₃ alkyl, Z is 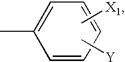
X₁ and Y are each selected from the group consisting of hydrogen, fluoro, chloro, bromo, trifluoromethyl, C₁-C₃ alkoxy and cyano, with the proviso that at least one of X and Y is other than hydrogen,
W is selected from the group consisting of hydrogen, fluoro, chloro, bromo, trifluoromethyl and alkoxy of from 1 to 3 carbon atoms,
and NR₄R₅ and Z have a cis relationship.

Compounds of Structure I may be prepared, for example, by the pathway shown in U.S. Pat. No. 4,536,518, incorporated by reference herein. An exemplary compound of Structure I is sertraline, (1S-cis)-4-(3,4-dichlorophenyl)-1,2,3,4-tetrahydro-N-methyl-1-naphthalenamine, which may be prepared as described in U.S. Pat. No. 4,536,518. Exemplary monoamine reuptake inhibitors that may be used in accordance with this invention also include, but are not limited to: femoxetine, which may be prepared as described in U.S. Pat. No. 3,912,743; fluoxetine, which may be prepared as described in U.S. Pat. No. 4,314,081; fluvoxamine, which may be prepared as described in U.S. Pat. No. 4,085,225; indalpine, which may be prepared as described in U.S. Pat. No. 4,064,255; indeloxazine, which may be prepared as described in U.S. Pat. No. 4,109,088; milnacipran, which may be prepared as described in U.S. Pat. No. 4,478,836; paroxetine, which may be prepared as described in U.S. Pat. No. 3,912,743 or U.S. Pat. No. 4,007,196; sibutramine, which may be prepared as described in U.S. Pat. No. 4,929,629; zimeldine, which may be prepared as described in U.S. Pat. No. 3,928,369; citalopram; and escitalopram. The patents cited herein are incorporated by reference herein. In an exemplary embodiment of this invention, the selective serotonin reuptake inhibitor is sertraline. In another exemplary embodiment of this invention, the selective serotonin reuptake inhibitor is fluoxetine. In another exemplary embodiment of this invention, the selective serotonin reuptake inhibitor is fluvoxamine.

The dopamine agonist of formula II may be prepared as described in U.S. Pat. No. 5,273,975, incorporated by reference herein.

Exemplary compounds of formula II include compounds wherein D is O, N—CH₃, NH, CH₂, or C═O.

Exemplary compounds of formula II also include compounds wherein A is SO₂, N, CH₂, CHCH₃, C═O, C═S, or C═NH.

Exemplary compounds of formula II also include compounds wherein B is CH₂, C═O, NH or N—CH₃.

Exemplary compounds of formula II also include compounds wherein D is O and A is CH₂, CHCH₃, C═O, C═S, or C═NH.

Exemplary compounds of formula II also include:
5-(Dimethylamino)-5,6-dihydro-4H-imidazo(4,5,1-ij)quinolin-2 (1H)-one hydrochloride;
5-(Propylamino)-5,6-dihydro-4H-imidazo(4,5,1-ij)quinolin-2 (1H)-one;
5-(Dipropylamino)-5,6-dihydro-4H-imidazo(4,5,1-ij)quinolin-2 (1H)-one;
(−)-5-(Dipropylamino)-5,6-dihydro-4H-imidazo(4,5,1-ij)quinolin-2 (1H)-one;
5,6-Dihydro-N,N-dipropyl-4H-imidazo(4,5,1-ij)quinolin-5-amine;
5,6-Dihydro-N-propyl-4H-imidazo(4,5,1-ij)quinoline-5-amine;
5,6-Dihydro-5-(dipropylamino)-4H-imidazo(4,5,1-ij)quinolin-8-ol;
5-(Dipropylamino)-5,6-dihydro-2H,4H-oxazolo(5,4,3-ij)quinolin-2-one;
5-(Propylamino)-5,6-dihydro-2H,4H-oxazolo(5,4,3-ij)quinolin-2-one;
5-(Dimethylamino)-5,6-dihydro-2H,4H-oxazolo(5,4,3-ij)quinolin-2-one;
(R)-5,6-dihydro-5-(methylamino)-4H-imidazo[4,5-ij]-quinolin-2 (1H)-one;
(R)-5,6-dihydro-5-(methyl-amino)-4H-imidazo[4,5-ij]-quinoline-2 (1H)-thione;
5,6-Dihydro-N,N-dipropyl-4H-pyrrolo(3,2,1-ij)quinolin-5-amine;
5,6-Dihydro-N,N-dimethyl-4H-pyrrolo(3,2,1-ij)quinolin-5-amine;
5,6-Dihydro-N,N-dipropyl-4H-pyrazolo(4,3,2-ij)quinolin-5-amine;
5,6-Dihydro-N,N-dimethyl-4H-pyrazolo(4,3,2-ij)quinolin-5-amine;
1,2,5,6-Tetrahydro-N,N-dipropyl-4H-pyrrolo(3,2,1-ij)quinolin-5-amine;
1,2,5,6-Tetrahydro-N,N-dimethyl4H-pyrrolo(3,2,1-ij)quinolin-5-amine;
5-(Propylamino)-5,6-dihydro-4H-pyrrolo(3,2,1-ij)quinolin-2,3-dione;
5-(Dipropylamino)-5,6-dihydro-4H-pyrrolo(3,2,1-ij)quinolin-2,3-dione;
5-(Propylamino)-5,6-dihydro-4H-pyrrolo(3,2,1-ij)quinolin-2 (1H)-one; and
5-(Dipropylamino)-5,6-dihydro-4H-pyrrolo(3,2,1-ij)quinolin-2 (1H)-one.

Dopamine agonists which may be used in the composition of the present invention may also include any selective D2/D3 agonists and nonselective dopamine agonists, which may include dopamine/β-adrenergic receptor agonists; dopamine/opiate receptor agonists; and dopamine agonists/α₂-adrenergic receptor antagonists. As used herein, “dopamine/β-adrenergic receptor agonists” are compounds that act as both dopamine agonists and as β-adrenergic receptor agonists; “dopamine/opiate receptor agonists” are compounds that act as both dopamine agonists and as opiate receptor agonists; and “dopamine agonists/α₂-adrenergic receptor antagonists” are compounds that act as both dopamine agonists and as α₂-adrenergic receptor antagonists. Exemplary dopamine agonists which may be used in accordance with the present invention include pramipexole, bromocriptine, lysuride, pergolide, aripiprazole and cabergoline.

The compounds used in the present invention may have optical centers and therefore may occur in different enantiomeric configurations. The compounds used in the present invention include all enantiomers, diastereomers, and other stereoisomers of the compounds, as well as racemic and other mixtures thereof. Individual isomers can be obtained by known methods, such as optical resolution, optically selective reaction, or chromatographic separation in the preparation of the final product or its intermediate.

The combination of a monoamine reuptake inhibitor or a pharmaceutically acceptable salt thereof and a dopamine agonist of formula II or pharmaceutically acceptable salt thereof is also referred to herein as “the active combination.” The active combination is a useful psychotherapeutic and may be used in the treatment of the disorders or conditions described herein, including without limitation those the treatment of which is facilitated by enhanced monoaminergic neurotransmission. Examples of the disorders or conditions which may be treated by the methods, compositions and kits of this invention are as follows:
depression, including, for example, depression in cancer patients, depression in Parkinson's patients, Postmyocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, bipolar depression BP I, or bipolar depression BP II; in an exemplary embodiment of this invention, depression is DSM-IV major depression, treatment-refractory major depression, bipolar depression BP I, or bipolar depression BP II; phobias, including, for example, agoraphobia, social phobia or simple phobias; eating disorders, including, for example, anorexia nervosa or bulimia nervosa; chemical dependencies, including, for example, addictions to alcohol, cocaine, amphetamine and other psychostimulants, morphine, heroin and other opioid agonists, phenobarbital and other barbiturates, nicotine, and diazepam and other benzodiazepines; memory disorders, including, for example, dementia, amnestic disorders, or age-related cognitive decline (ARCD); Parkinson's diseases, including, for example, dementia in Parkinson's disease, neuroleptic-induced parkinsonism or tardive dyskinesias; dementia in patients with human immunodeficiency virus (HIV), an endocrine disorder, including, for example, hyperprolactinaemia; vasospasm, including, for example, vasospasm in the cerebral vasculature; gastrointestinal tract disorders, including, for example, gastrointestinal tract disorders involving changes in motility and secretion; cancer, including, for example, small cell lung carcinoma; and headache, including, for example, headache associated with vascular disorders.

As used herein, “mammal” means any member of the class Mammalia. As an example, the mammal in need of the treatment may be a human. As another example, the mammal in need of the treatment may be a mammal other than a human.

A dopamine agonist of formula II and a monoamine reuptake inhibitor, each of which is used in formulating the pharmaceutical composition of this invention, are each referred to herein as an “active compound.” An active compound which is basic in nature is capable of forming a wide variety of different salts, for example with various inorganic and organic acids. The acid addition salts are readily prepared by treating the base compounds with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the active compounds used in formulating the pharmaceutical composition of this invention that are basic in nature are those which form non-toxic acid addition salts, i.e., salts containing pharmacologically acceptable anions, such as bisulfate, acid phosphate, fumarate, citrate, acid citrate, bitartrate, gluconate, saccharate, hydrochloride, hydrobromide, hydroiodide, sulfate, phosphate, acetate, propionate, lactate, maleate, malate, succinate, tartrate, cyclohexanesulfamates, methanesulfonates, ethanesulfonates, benzenesulfonates, toluenesulfonates and other pharmaceutically acceptable counter ions for amines.

The dopamine agonists of formula II and monoamine reuptake inhibitors used in formulating the pharmaceutical composition of this invention are preferably administered together as part of a pharmaceutical composition. For example, compositions containing the monoamine reuptake inhibitors and the dopamine agonists of formula II can be administered as solutions in a volume per body weight of 1 ml/kg. The vehicle used is varied depending on the solubility of the monoamine reuptake inhibitor and of the dopamine agonist used. The dopamine agonist of formula II and monoamine reuptake inhibitor may also be administered separately, either simultaneously or in various sequences. For example, each active compound may be part of a pharmaceutical composition, wherein the pharmaceutical composition containing the dopamine agonist and the pharmaceutical composition containing the monoamine reuptake inhibitor may be administered separately. The dopamine agonist of formula II and the monoamine reuptake inhibitor may be administered in either order, provided that after administration of the first active compound, the second active compound is administered within 12 hours or less.

The dopamine agonists of formula II and the monoamine reuptake inhibitors used in formulating the pharmaceutical composition of this invention may advantageously be used in conjunction with other therapeutic agents which do not appreciably block monoamine uptake or affect monoamine oxidase, such as mirtazapine, mianserin, bupropion, lithium salts, antiepileptic drugs such as caramazepine, valproate, lamotrigine, topiramate, gabapentin, pregabalin, and/or with antiparkinsonian agents such as dopaminergic antiparkinsonian agents such as levodopa, preferably in combination with a peripheral decarboxylase inhibitor such as benserazide or carbidopa. It is to be understood that the present invention covers the use of a monoamine reuptake inhibitor or a pharmaceutically acceptable salt thereof and a dopamine agonist of formula II or pharmaceutically acceptable salt thereof in combination with one or more other therapeutic agents.

Activity of the active combinations as antidepressants and related pharmacological properties can be determined by methods (1)-(3) below, which are described in Koe, B. et al. Journal of Pharmacology and Experimental Therapeutics, 226 (3), 686-700 (1983). Specifically, activity can be determined by studying (1) their ability to affect the efforts of mice to escape from a swim-tank by the Porsolt mouse “behavior despair” test, (2) their ability to potentiate 5-hydroxytryptophan-induced behavioral symptoms in mice in vivo, and (3) their ability to block the uptake of serotonin, norepinephrine, dopamine or a combination thereof by synaptosomal rat brain cells in vitro. The ability of the active combinations to counteract reserpine hypothermia in mice in vivo can be determined according to the methods described in U.S. Pat. No. 4,029,731. The activity of the active combinations as antidepressants and related pharmacological properties also can be determined by methods (4)-(8) below. Specifically, activity can be determined by studying (4) their ability to reverse the stress-induced decrease in sucrose intake in rodents described in Papp, M. et al., European Journal of Pharmacology, 261, 141-147 (1994), (5) learned helplessness paradigm described in Martin P et al., Life Sciences, 48, 2505-2511 (1991), (6) reversing the behavioral deficits of olfactory bulbectomized rats described in Broekkamp CL et al., Pharmacology, Biochemistry and Behavior, 13, 643-646 (1980), (7) increasing down-regulation or desensitization of beta-adrenergic receptors described in Mishra R. et al., Neuropharmacology, 19, 983-987 (1980), and (8) increasing extracellular levels of serotonin, norepinephrine, and/or dopamine in the prefrontal cortex of freely-moving rodents by in vivo dialysis described in Millan M J et al., European Journal of Neuroscience, 12, 1079-1095 (2000).

The pharmaceutical compositions described herein may be prescription pharmaceutical compositions or over-the-counter pharmaceutical compositions. As used herein, a “prescription pharmaceutical composition” is a composition which is effective to deliver an active compound to a human as prescribed by a physician. An “over-the-counter pharmaceutical composition” is a composition which is effective to deliver an active compound to a human which does not require a prescription from a physician in order to be administered to the human.

The pharmaceutical composition described herein may be formulated in a conventional manner using one or more pharmaceutically acceptable carriers. Thus, the active combinations of this invention may be formulated for oral, buccal, intranasal, parenteral (for example, intravenous, intramuscular or subcutaneous) or rectal administration or in a form suitable for administration by inhalation or insufflation, and may be administered orally, buccally, intranasally, parenterally (for example, intravenously, intramuscularly or subcutaneously) or rectally or by inhalation or insufflation.

For oral administration, the pharmaceutical composition may take the form of, for example, tablets or capsules prepared by conventional means with pharmaceutically acceptable excipients such as binding agents, including pregelatinized maize starch, polyvinylpyrrolidone or hydroxypropyl methylcellulose; fillers, including lactose, microcrystalline cellulose or calcium phosphate; lubricants, including magnesium stearate, talc or silica; disintegrants, including potato starch or sodium starch glycolate; or wetting agents, including sodium lauryl sulphate. The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, including sorbitol syrup, methyl cellulose or hydrogenated edible fats; emulsifying agents, including lecithin or acacia, non-aqueous vehicles, including almond oil, oily esters or ethyl alcohol; and preservatives, including methyl or propyl p-hydroxybenzoates or sorbic acid.

For buccal administration, the composition may take the form of tablets or lozenges formulated in conventional manner.

The active compounds used in formulating the pharmaceutical composition of this invention may be formulated for parenteral administration by injection, including using conventional catheterization techniques or infusion. Formulations for injection may be presented in unit dosage form, for example, in ampoules or in multi-dose containers, with an added preservative.

The composition containing the active compounds may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, for example, sterile pyrogen-free water, before use.

The active compounds used in formulating the pharmaceutical composition of this invention may also be formulated in rectal compositions such as suppositories or retention enemas, for example, containing conventional suppository bases such as cocoa butter or other glycerides.

For intranasal administration or administration by inhalation, the active compounds used in formulating the pharmaceutical composition of this invention are conveniently delivered in the form of a solution or suspension from a pump spray container that is squeezed or pumped by the patient or as an aerosol spray presentation from a pressurized container or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the unit dose may be determined by providing a valve to deliver a metered amount. The pressurized container or nebulizer may contain a solution or suspension of the active compounds. Capsules and cartridges, made, for example, from gelatin, for use in an inhaler or insufflator may be formulated containing a powder mix of a compound of this invention and a suitable powder base such as lactose or starch.

The dopamine agonists of formula II and the monoamine reuptake inhibitors used in formulating the pharmaceutical composition of this invention may be administered alone or preferably together with pharmaceutically acceptable carriers by any of the routes previously indicated, and such administration may be carried out in both single and multiple doses. More particularly, the active combination can be administered in a wide variety of different dosage forms, i.e., the active combination may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, oral pharmaceutical formulations containing the active combination may be suitably sweeteed and/or flavored by means of various agents of the type commonly employed for such purposes.

In one embodiment of the invention, the amounts of a) the monoamine reuptake inhibitor or pharmaceutically acceptable salt thereof, and b) the dopamine agonist of formula II or pharmaceutically acceptable salt thereof may be amounts such that the combination of a) and b) is effective in treating the disorder or condition. In another embodiment of the invention, the amount of the monoamine reuptake inhibitor or pharmaceutically acceptable salt thereof may be an amount effective in enhancing monoaminergic neurotransmission in a mammal.

An exemplary daily dose of a monoamine reuptake inhibitor in a pharmaceutical composition of this invention for oral, parenteral, rectal or buccal administration to the average adult human for the treatment of the conditions referred to above is from about 1 mg to about 300 mg of monoamine reuptake inhibitor per unit dose administered 1 to 3 times per day, such as about 25 mg to about 300 mg of sertraline, preferably from about 50 mg to about 200 mg of sertraline per unit dose which could be administered, for example 1 to 3 times per day, or such as about 10 mg to about 40 mg of fluoxetine per unit dose which could be administered, for example 1 to 3 times per day, or such as about 100 mg to about 200 mg of fluvoxamine per unit dose which could be administered, for example 1 to 3 times per day. Exemplary and preferred doses for other monoamine reuptake inhibitors are determined on a compound by compound basis. An exemplary daily dose of the dopamine agonist of formula II in a pharmaceutical composition of this invention for oral, parenteral, rectal or buccal administration to the average adult human for the treatment of the conditions referred to above is from about 0.1 to about 100 mg of dopamine agonist per unit dose administered 1 to 3 times per day, such as from about 1 mg to about 50 mg of 5-(Dipropylamino)-5,6-dihydro-2H,4H-oxazolo(5,4,3-ij)quinolin-2-one, 5-(Propylamino)-5,6-dihydro-2H,4H-oxazolo(5,4,3-ij)quinolin-2-one, 5-(Dimethylamino)-5,6-dihydro-2H,4H-oxazolo(5,4,3-ij)quinolin-2-one, (R)-5,6-dihydro-5-(methylamino)-4H-imidazo[4,5-ij]-quinolin-2 (1H)-one, or (R)-5,6-dihydro-5-(methyl-amino)-4H-imidazo[4,5-ij]-quinoline-2 (1H)-thione per unit dose which could be administered, for example 1 to 3 times per day.

Other dopamine agonists may be used including, for example, from about 1 mg to about 10 mg of cabergoline per unit dose which could be administered, for example 1 to 3 times per day, and from 0.1 mg to about 2 mg of pramipexole, preferably from about 0.25 mg to about 1.5 mg of pramipexole per unit dose which could be administered, for example 1 to 3 times per day.

An exemplary dose ratio by weight of a monoamine reuptake inhibitor to a dopamine agonist of formula II combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in a mammal, such as an average adult human are preferably arranged so that each metered dose or “puff” of aerosol contains from about 100 μg to about 30,000 μg of the dopamine agonist of formula II, preferably from about 250 μg to about 1,000 μg of dopamine agonist of formula II, and from about 1,000 μg to about 30,000 μg of a monoamine reuptake inhibitor, preferably from about 5,000 μg to about 20,000 μg. Administration may be once or several times daily, for example 1, 3, 4 or 8 times, giving for example, 1, 2 or 3 doses each time.

The present invention also relates to combining separate pharmaceutical compositions in a kit. The kit may comprise two separate pharmaceutical compositions, wherein a first pharmaceutical composition contains a dopamine agonist of formula II or pharmaceutically acceptable salt thereof, and a second pharmaceutical composition contains a monoamine reuptake inhibitor or pharmaceutically acceptable salt thereof. Each composition may be in the form of at least one unit dosage form. Alternatively, the kit may comprise a pharmaceutical composition containing a dopamine agonist of formula II or pharmaceutically acceptable salt thereof and a monoamine reuptake inhibitor or pharmaceutically acceptable salt thereof. The composition may be in the form of at least one dosage form comprising at least one unit dose of dopamine agonist of formula II and at least one dose of monoamine reuptake inhibitor. Preferably, the compositions in the kit comprise a pharmaceutically acceptable carrier.

The kit comprising two separate pharmaceutical composition may comprise a container for containing the two separate compositions. The containers are those known in the art, including, for example, a bottle or foil packet, and may have a separate containing area for each of the two pharmaceutical compositions. The kit may also comprise directions for the administration of the separate components. The kit form is advantageous when the dopamine agonist of formula II and the monoamine reuptake inhibitor are administered in different dosage forms, such as, for example, oral and parenteral form, or when the dopamine agonist of formula II and the monoamine reuptake inhibitor are administered at different dosage intervals, or when separate titration of the dopamine agonist of formula II and the monoamine reuptake inhibitor of the combination is desirable.

An example of such a kit is a so-called blister pack. Blister packs are commonly used for the packaging of pharmaceutical unit dosage forms such as tablets or capsules. The kit may also comprise a memory aid for a patient on a regimen, where the memory aid may be, for example, in the form of numbers where each number is next to a tablet or capsule to be ingested on the day of the regimen corresponding to the number. The memory aid may also be, for example, in the form of a calendar printed on a card, where each calendar day indicates the amount of each active compound corresponding to a daily dose for that day, where a daily dose may be a single tablet or capsule or several pills or capsules to be taken on a given day. Also, a daily dose of dopamine agonist of formula II can consist of one tablet or capsule while a daily dose of the monoamine reuptake inhibitor can consist of several tablets or capsules and vice versa. Other variations of memory aids will be readily apparent. The kit may also comprise a dispenser designed to dispense daily doses in the order of their intended use. The dispenser may be equipped with a memory-aid to facilitate compliance with the regimen. An example of such a memory-aid is a mechanical counter indicating the number of daily doses that have been dispensed. Another example of such a memory-aid is a battery-powered micro-chip memory coupled with a liquid crystal readout, or audible reminder signal which, for example, reads out the date that the last daily dose has been taken and/or reminds the patient when the next dose is to be taken.

It should be understood that the present invention is not limited to the embodiments described herein. Numerous modifications can be made by one skilled in the art having the benefits of the teachings given here. Such modifications should be taken as being encompassed within the scope of the present invention as set forth in the appended claims.

1. A pharmaceutical composition, comprising: (i) a dopamine agonist of formula 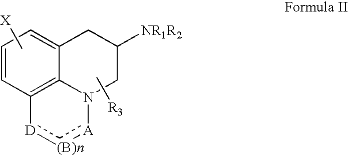
or a pharmaceutically acceptable salt of said dopamine agonist,
wherein R₁, R₂, and R₃ are each independently hydrogen, C₁₋₆ alkyl, C₂₋₆ alkenyl, or C₂₋₆ alkynyl, C₃₋₁₀ cycloalkyl, or R₁ and R₂ are joined to form together with the nitrogen of NR₁R₂ a C₃₋₇ cyclic amine which can contain in addition to said nitrogen one or more heteroatoms selected from the group consisting of N, S and O; X is hydrogen, C₁₋₆ alkyl, halogen, hydroxy, C₁₋₆ alkoxy, cyano, carboxamide, carboxyl, or C₁-C₆ alkoxycarbonyl; A is SO₂, N, CH, CH₂, CHCH₃, C═O, C═S, CSCH₃, C═NH, CNH₂, CNHCH₃, CNHCOOCH₃, or CNHCN; and B is CH₂, CH, C═O, N, NH or N—CH₃; n is 0 or 1; and D is CH, CH₂, C═O, O, N, NH or N—CH₃; (ii) a monoamine reuptake inhibitor or pharmaceutically acceptable salt thereof; and optionally (iii) a pharmaceutically acceptable carrier.

2. The composition of claim 1, wherein the monoamine reuptake inhibitor is a compound having the formula 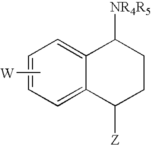
or a pharmaceutically acceptable salt thereof,
wherein R₄ is selected from the group consisting of hydrogen and C₁-C₃ alkyl, R₅ is C₁-C₃ alkyl, Z is 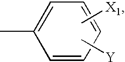
X₁ and Y are each selected from the group consisting of hydrogen, fluoro, chloro, bromo, trifluoromethyl, C₁-C₃ alkoxy and cyano, with the proviso that at least one of X and Y is other than hydrogen,
W is selected from the group consisting of hydrogen, fluoro, chloro, bromo, trifluoromethyl and alkoxy of from 1 to 3 carbon atoms,
and NR₄R₅ and Z have a cis relationship.

3. The pharmaceutical composition of claim 1, wherein the monoamine reuptake inhibitor is selected from the group consisting of femoxetine, fluoxetine, fluvoxamine, indalpine, indeloxazine, milnacipran, paroxetine, sertraline, sibutramine, zimeldine, citalopram, escitalopram, imipramine, desiramine, clomipramine, maprotiline, reboxetine, duloxetine, atomoxetine, nefazodone and trazodone.

4. The pharmaceutical composition of claim 3, wherein the monoamine reuptake inhibitor is selected from the group consisting of sertraline, fluoxetine, and fluvoxamine.

5. The pharmaceutical composition of claim 1, wherein D is O, N—CH₃, NH, CH₂, or C═O.

6. The pharmaceutical composition of claim 1, wherein A is SO₂, N, CH₂, CHCH₃, C═O, C═S, or C═NH.

7. The pharmaceutical composition of claim 1, wherein B is CH₂, C═O, NH or N—CH₃.

8. The pharmaceutical composition of claim 1, wherein D is 0 and A is CH₂, CHCH₃, C═O, C═S, or C═NH.

9. The pharmaceutical composition of claim 1, wherein the compound of formula II is selected from the group consisting of
5-(Dimethylamino)-5,6-dihydro-4H-imidazo(4,5,1-ij)quinolin-2 (1H)-one hydrochloride;
5-(Propylamino)-5,6-dihydro-4H-imidazo(4,5,1-ij)quinolin-2 (1H)-one;
5-(Dipropylamino)-5,6-dihydro-4H-imidazo(4,5,1-ij)quinolin-2 (1H)-one;
(−)-5-(Dipropylamino)-5,6-dihydro-4H-imidazo(4,5,1-ij)quinolin-2 (1H)-one;
5,6-Dihydro-N,N-dipropyl-4H-imidazo(4,5,1-ij)quinolin-5-amine;
5,6-Dihydro-N-propyl-4H-imidazo(4,5,1-ij)quinoline-5-amine;
5,6-Dihydro-5-(dipropylamino)-4H-imidazo(4,5,1-ij)quinolin-8-ol;
5-(Dipropylamino)-5,6-dihydro-2H,4H-oxazolo(5,4,3-ij)quinolin-2-one;
5-(Propylamino)-5,6-dihydro-2H,4H-oxazolo(5,4,3-ij)quinolin-2-one;
5-(Dimethylamino)-5,6-dihydro-2H,4H-oxazolo(5,4,3-ij)quinolin-2-one;
(R)-5,6-dihydro-5-(methylamino)-4H-imidazo[4,5-ij]-quinolin-2 (1H)-one;
(R)-5,6-dihydro-5-(methyl-amino)-4H-imidazo[4,5-ij]-quinoline-2 (1H)-thione;
5,6-Dihydro-N,N-dipropyl-4H-pyrrolo(3,2,1-ij)quinolin-5-amine;
5,6-Dihydro-N,N-dimethyl-4H-pyrrolo(3,2,1-ij)quinolin-5-amine;
5,6-Dihydro-N,N-dipropyl-4H-pyrazolo(4,3,2-ij)quinolin-5-amine;
5,6-Dihydro-N,N-dimethyl-4H-pyrazolo(4,3,2-ij)quinolin-5-amine;
1,2,5,6-Tetrahydro-N,N-dipropyl-4H-pyrrolo(3,2,1-ij)quinolin-5-amine;
1,2,5,6-Tetrahydro-N,N-dimethyl-4H-pyrrolo(3,2,1-ij)quinolin-5-amine;
5-(Propylamino)-5,6-dihydro-4H-pyrrolo(3,2,1-ij)quinolin-2,3-dione;
5-(Dipropylamino)-5,6-dihydro-4H-pyrrolo(3,2,1-ij)quinolin-2,3-dione;
5-(Propylamino)-5,6-dihydro-4H-pyrrolo(3,2,1-ij)quinolin-2 (1H)-one; and
5-(Dipropylamino)-5,6-dihydro-4H-pyrrolo(3,2,1-ij)quinolin-2 (1H)-one.

10. The pharmaceutical composition of claim 9, wherein the compound of formula II is selected from the group consisting of
5-(Dipropylamino)-5,6-dihydro-2H,4H-oxazolo(5,4,3-ij)quinolin-2-one;
5-(Propylamino)-5,6-dihydro-2H,4H-oxazolo(5,4,3-ij)quinolin-2-one;
5-(Dimethylamino)-5,6-dihydro-2H,4H-oxazolo(5,4,3-ij)quinolin-2-one;
(R)-5,6-dihydro-5-(methylamino)-4H-imidazo[4,5-ij]-quinolin-2 (1H)-one; and
(R)-5,6-dihydro-5-(methyl-amino)-4H-imidazo[4,5-ij]-quinoline-2 (1H)-thione;

11. The pharmaceutical composition of claim 10, wherein the monoamine reuptake inhibitor is selected from the group consisting of sertraline, fluoxetine, and fluvoxamine.

12. The pharmaceutical composition of claim 1, wherein a) the monoamine reuptake inhibitor or pharmaceutically acceptable salt thereof and b) the dopamine agonist of formula II or pharmaceutically acceptable salt thereof are present in amounts such that the combination of a) and b) is effective in treating a disorder or condition selected from the group consisting of hypertension, depression, generalized anxiety disorder, phobias, posttraumatic stress syndrome, avoidant personality disorder, sexual dysfunction, eating disorders, obesity, chemical dependencies, cluster headache, pain, Alzheimer's disease, obsessive-compulsive disorder, panic disorder, memory disorders, Parkinson's diseases, endocrine disorders, vasospasm, cerebellar ataxia, gastrointestinal tract disorders, negative symptoms of schizophrenia, premenstrual syndrome, Fibromyalgia Syndrome, stress incontinence, Tourette's syndrome, trichotillomania, kleptomania, male impotence, cancer, chronic paroxysmal hemicrania, headache and a combination thereof in a mammal.

13. The pharmaceutical composition of claim 1, wherein the monoamine reuptake inhibitor or pharmaceutically acceptable salt thereof is present in an amount effective in enhancing monoaminergic neurotransmission in a mammal.

14. The pharmaceutical composition of claim 12, wherein the disorder or condition is depression selected from the group consisting of DSM-IV major depression, treatment-refractory major depression, bipolar depression BP I, or bipolar depression BP II.

15. A method for treating a disorder or condition selected from the group consisting of hypertension, depression, generalized anxiety disorder, phobias, posttraumatic stress syndrome, avoidant personality disorder, sexual dysfunction, eating disorders, obesity, chemical dependencies, cluster headache, pain, Alzheimer's disease, obsessive-compulsive disorder, panic disorder, memory disorders, Parkinson's diseases, endocrine disorders, vasospasm, cerebellar ataxia, gastrointestinal tract disorders, negative symptoms of schizophrenia, premenstrual syndrome, Fibromyalgia Syndrome, stress incontinence, Tourette's syndrome, trichotillomania, kleptomania, male impotence, cancer, chronic paroxysmal hemicrania, headache and a combination thereof in a mammal, comprising administering to a mammal in need of such treatment (i) a dopamine agonist of formula II as described in claim 1 or a pharmaceutically acceptable salt of said dopamine agonist; and (ii) a monoamine reuptake inhibitor or a pharmaceutically acceptable salt of said reuptake inhibitor.

16. The method of claim 15, wherein the dopamine agonist is administered in unit doses each comprising about 0.1 to about 100 mg of the dopamine agonist, and the monoamine reuptake inhibitor is administered in unit doses each comprising about 1 to about 300 mg of the monoamine reuptake inhibitor.

17. The method of claim 16, wherein a unit dose of the dopamine agonist and a unit dose of the monoamine reuptake inhibitor are administered one to three times per day.

18. The method of claim 15, wherein the dopamine agonist is selected from the group consisting of
5-(Dipropylamino)-5,6-dihydro-2H,4H-oxazolo(5,4,3-ij)quinolin-2-one;
5-(Propylamino)-5,6-dihydro-2H,4H-oxazolo(5,4,3-ij)quinolin-2-one;
5-(Dimethylamino)-5,6-dihydro-2H,4H-oxazolo(5,4,3-ij)quinolin-2-one;
(R)-5,6-dihydro-5-(methylamino)-4H-imidazo[4,5-ij]-quinolin-2 (1H)-one; and
(R)-5,6-dihydro-5-(methyl-amino)-4H-imidazo[4,5-ij]-quinoline-2 (1H)-thione;

19. The method of claim 15, wherein the monoamine reuptake inhibitor is selected from the group consisting of sertraline, fluoxetine, and fluvoxamine.

20. The method of claim 19, wherein the dopamine agonist is selected from the group consisting of
5-(Dipropylamino)-5,6-dihydro-2H,4H-oxazolo(5,4,3-ij)quinolin-2-one;
5-(Propylamino)-5,6-dihydro-2H,4H-oxazolo(5,4,3-ij)quinolin-2-one;
5-(Dimethylamino)-5,6-dihydro-2H,4H-oxazolo(5,4,3-ij)quinolin-2-one;
(R)-5,6-dihydro-5-(methylamino)-4H-imidazo[4,5-ij]-quinolin-2 (1H)-one; and
(R)-5,6-dihydro-5-(methyl-amino)-4H-imidazo[4,5-ij]-quinoline-2 (1H)-thione;

21. A method for treating a disorder or condition that can be treated by enhancing monoaminergic neurotransmission in a mammal, comprising administering to a mammal in need of such treatment (i) a dopamine agonist of formula II as described in claim 1 or a pharmaceutically acceptable salt of said dopamine agonist; and (ii) a monoamine reuptake inhibitor or a pharmaceutically acceptable salt of said reuptake inhibitor.

22. A method for enhancing monoaminergic neurotransmission in a mammal, comprising administering to a mammal in need of such treatment (i) a dopamine agonist of formula II as described in claim 1 or a pharmaceutically acceptable salt of said dopamine agonist; and (ii) a monoamine reuptake inhibitor or a pharmaceutically acceptable salt of said reuptake inhibitor.

23. A pharmaceutical composition consisting essentially of: (i) a dopamine agonist of formula II as described in claim 1 or a pharmaceutically acceptable salt of said dopamine agonist; (ii) a selective serotonin reuptake inhibitor or a pharmaceutically acceptable salt of said selective serotonin reuptake inhibitor; and optionally (iii) a pharmaceutically acceptable carrier.

24. A kit comprising:
a) a dopamine agonist unit dosage form comprising a dopamine agonist of formula II as described in claim 1 or a pharmaceutically acceptable salt of said dopamine agonist and a pharmaceutically acceptable carrier;
b) a monoamine reuptake inhibitor unit dosage form comprising a monoamine reuptake inhibitor or a pharmaceutically acceptable salt of said reuptake inhibitor and a pharmaceutically acceptable carrier; and optionally
c) a container.

25. A kit comprising:
a) at least one unit dosage form comprising a unit dose of a dopamine agonist of formula II as described in claim 1 or a pharmaceutically acceptable salt of said dopamine agonist, a unit dose of a monoamine reuptake inhibitor or a pharmaceutically acceptable salt of said reuptake inhibitor, and a pharmaceutically acceptable carrier; and optionally
b) a container.
